(19) United States
(12) Patent Application Publication
(10) Pub. No.: US 2026/0191471 A1
(43) Pub. Date: Jul. 9, 2026

(54) METHOD AND SYSTEM FOR CONFIRMATION OF MICRONEEDLE-BASED ANALYTE-SELECTIVE SENSOR INSERTION INTO VIABLE TISSUE VIA ELECTRICAL INTERROGATION

(71) Applicant: Biolinq Incorporated, San Diego, CA (US)

(72) Inventors: Joshua WINDMILLER, San Diego, CA (US); Jared Rylan TANGNEY, Encinitas, CA (US)

(21) Appl. No.: 19/557,038

(22) Filed: Mar. 4, 2026

Related U.S. Application Data
(63) Continuation of application No. 16/051,398, filed on Jul. 31, 2018, now Pat. No. 12,599,337.
(60) Provisional application No. 62/542,774, filed on Aug. 8, 2017.

(51) Int. Cl. A61B 5/00; A61B 5/0531; A61B 5/145; C12Q 1/00; G01N 27/327; G16H 40/40

(52) U.S. Cl. CPC A61B 5/685; A61B 5/14532; A61B 5/14546; C12Q 1/001; G01N 27/327; G01N 27/3271; G16H 40/40; A61B 5/0531; A61B 2562/0295

(57) ABSTRACT

A system and method to confirm the insertion of an analyte-selective sensor comprising an array of microneedles possessing vertical extent between 200 and 2000 μm into viable tissue is disclosed herein. Mechanical insertion of an analyte-selective sensor is firstly attempted by means of an application of external force. Either during or following this routine, an electrical stimulus is applied between at least two distinct electrodes located within the said analyte-selective sensor; a resultant response is measured. This response is compared with a reference value to determine if insertion was successful. If insertion was successful, no further effort is required and the sensor can operate as intended. However, if insertion was not successful, the user can be instructed to continue to apply additional force to said sensor to achieve successful insertion or otherwise re-apply the said sensor altogether.

### CROSS REFERENCE TO RELATED APPLICATION

The Present Application claims priority to U.S. Patent Application No. 62/542774, filed on Aug. 8, 2017, which is hereby incorporated by reference in its entirety.

### STATEMENT REGARDING FEDERALLY SPONSORED RESEARCH OR DEVELOPMENT

Not Applicable

BACKGROUND OF THE INVENTION

### Field of the Invention

The present invention generally relates to micro-needle sensors.

### Description of the Related Art

The ability to confirm the insertion of a microneedle-based analyte-selective sensor into viable tissue is of paramount importance in the electrochemical sensors domain. Although corroborating insertion of needle-based sensors is quite straightforward owing to the intrinsic macro-scale geometry of such devices and extent of penetration into tissue, as sensors are further miniaturized and penetration into viable tissues becomes more superficial, the assessment of proper insertion grows in importance. Often, when attempts are made to insert micron-scale analyte-selective sensors, such as microneedles, into viable tissues, this typically involves assessing if the absolute levels of signal generated by said sensors reside in a reasonable and expected range of values. Indeed, the ability to corroborate appropriate sensor insertion and persistence in viable tissue has posed a formidable challenge to those aiming to create intracutaneously-and subcutaneously-implanted micron-scale electrochemical sensors for the quantification of circulating analytes in physiological fluids.

In order to ensure proper operation of a microneedle-based analyte-selective sensor in vivo, successful insertion of said sensor must be corroborated, otherwise accurate readings may be compromised.

Prior art solutions have been concerned with visual inspection of analyte-selective sensor insertion-successful insertion of a macro-scale sensor is straightforward to observe. Such a technique, however, is not amenable as the size of said sensor scales to microscopic levels, as is the case with microneedle sensors, whose dimensions fall within 20-2000 μm. Under such circumstances, normal operation is instigated and it is often left to the user to determine if the ensuing measurement resides within an expected range.

Prior art solutions have also been concerned with the user's perception of pain or discomfort upon analyte-selective sensor insertion owing to the permeation of the nerve layer in the dermis, hypodermis, and muscle tissues. As these sensors are further miniaturized and insertion becomes increasingly superficial, the permeation of the nerve layer might be avoided entirely. Hence, even upon proper insertion, a pain or discomfort sensation might not be readily apparent.

Such prior art includes the following.

Brister et al., U.S. Pat. No. 7,905,833 for a Transcutaneous analyte sensor discloses systems and methods for measuring an analyte in a host. More particularly, the Brister relates to systems and methods for transcutaneous measurement of glucose in a host.

Hayter et al., U.S. Pat. No. 9,008,743 for a Method and apparatus for providing data processing and control in medical communication system, discloses methods and apparatus for providing data processing and control for use in a medical communication system are provided.

Angel et al., U.S. Pat. No. 7,645,263 for an Impedance Sensor, discloses a transdermal transport device includes a reservoir for holding a formulation of an active pharmaceutical ingredient, a needle with a bore through which the formulation is transported between the reservoir and a target area of a biological body, and an impedance sensor. The impedance sensor has an electrode positioned to measure the impedance of a portion of the target area between the needle and the electrode to indicate the depth of penetration of the needle into the target area.

Liang et al., U.S. Pat. No. 8,160,834 for Methods and systems for observing sensor parameters, discloses methods and materials for observing the state of a sensor, for example those used by diabetic patients to monitor blood glucose levels. Typically a voltage such as a voltage pulse is applied to the sensor in order to solicit a current response from which for example, factors such as impedance values can be derived. Such values can then be used as indicators of a sensor's state, for example the state of sensor hydration, sensor noise, sensor offset, sensor drift or the like.

### BRIEF SUMMARY OF THE INVENTION

The technology described herein relates to implantable, analyte-selective microneedle sensors and operation of the same.

The current invention teaches of a method for the identification of successful insertion of a microneedle-based analyte-selective sensor into a viable tissue, including the epidermis, dermis, and hypodermis. Mechanical insertion of an analyte-selective sensor is firstly attempted by means of an application of external force. Either during or following this routine, an electrical stimulus is applied between at least two distinct electrodes located within the said analyte-selective sensor; a resultant response is measured. This response is compared with a reference value to determine if insertion was successful. If insertion was successful, no further effort is required and the sensor can operate as intended. However, if insertion was not successful, the user can be instructed to continue to apply additional force to said sensor to achieve successful insertion or otherwise re-apply the said sensor altogether.

One aspect of the present invention is a method to confirm the insertion of an analyte-selective sensor comprising an array of microneedles possessing vertical extent between 200 and 2000 μm into viable tissue. The method includes attempting mechanical insertion of at least two spatially-distinct microneedle structures on said array, each featuring a single electrode, into a viable tissue. The method also includes applying a fixed or time-varying electrical potential or current to the at least two electrodes following said attempt at mechanical insertion. The method also includes measuring the resultant electrical potential, current, resistance, conductance, capacitance, or impedance value generated between the two electrodes and comparing said value to a known reference value to corroborate successful insertion and in situ access to said viable tissue.

Another aspect of the present invention is a method to confirm the insertion of a microneedle-based analyte-selective sensor into cutaneous tissue. The method includes attempting mechanical insertion of at least two electrodes comprising said analyte-selective sensor into the cutaneous tissue. The method also includes applying a fixed or time-varying electrical potential or current to the at least two electrodes following said attempt at mechanical insertion. The method also includes measuring the resultant electrical potential, current value generated between said two electrodes and comparing said value to a known reference value to corroborate successful insertion and in situ access to said cutaneous tissue.

Yet another aspect of the present invention is a method to activate a microneedle-based analyte-selective sensor by completing an electrical circuit upon the successful mechanical insertion of at least two spatially-distinct microneedle structures on said sensor, each microneedle constituent featuring a single electrode, into a viable tissue, thereby transitioning said sensor from an OFF/hibernate to ON/active state.

Yet another aspect of the present invention is a method to confirm the insertion of a microneedle-based analyte-selective sensor into cutaneous tissue. The method includes inserting into cutaneous tissue of a user at least two spatially-distinct microneedle structures of an array, each spatially-distinct microneedle structure comprising a single electrode and extending between 200 and 2000 μm from a base of the array. The method also includes applying a fixed or time-varying electrical potential or current to the at least two electrodes following the insertion into the cutaneous tissue. The method also includes measuring a resultant electrical potential, current value generated between the at least two electrodes. The method also includes comparing the value to a known reference value to corroborate successful insertion and in situ access to the cutaneous tissue of the user.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 illustrates an analyte-selective sensor featuring three microneedle structures, each measuring 1200 μm in vertical extent and designed to permeate the stratum corneum to access the interstitial fluid occupying the viable dermis.

FIG. 2 is an exploded view of a microneedle-based analyte-selective sensor system with the microneedle array and excitation and measurement circuit shown.

FIG. 2A is an expanded view of the microneedle constituents is provided in circle 2A of FIG. 2.

FIG. 3 is a cutaway view of a microneedle-based analyte-selective sensor system illustrating successful permeation/penetration of the stratum corneum (brown) to access the viable dermis (pink).

FIG. 4 is a block/process flow diagram illustrating the process flow to corroborate successful sensor insertion.

FIG. 5 is a diagrammatic representation of a decision tree delineating the assessment of tissue penetration and hence sensor insertion.

FIG. 5A is a diagrammatic representation of a decision tree delineating the assessment of tissue penetration and hence sensor insertion.

FIG. 5B is a diagrammatic representation of a decision tree delineating the assessment of tissue penetration and hence sensor insertion.

FIG. 5C is a diagrammatic representation of a decision tree delineating the assessment of tissue penetration and hence sensor insertion.

FIG. 6 is a diagrammatic representation of analyte-selective sensor featuring electrodes that have not successfully penetrated/permeated a biological interface/tissue, as determined by a measurement circuit.

FIG. 6A is a diagrammatic representation of analyte-selective sensor featuring electrodes that have successfully penetrated/permeated a biological interface/tissue, as determined by a measurement circuit.

### DETAILED DESCRIPTION OF THE INVENTION

The current invention represents a simple and straightforward approach facilitating the confirmation of a successful sensor insertion event; the disclosed technique addresses the shortcomings of the prior art while remaining amenable to established methods of applying intracutaneous and intradermal analyte-selective electrochemical sensors.

In order to tender accurate physiological or physiochemical readings of a particular analyte or group of analytes, an analyte-selective electrochemical sensor must remain in fluidic contact with the tissue, physiological fluid, or physiological compartment of interest and, often, the active sensing region of said electrochemical sensor must maintain full immersion or coverage within the said tissue, physiological fluid, or physiological compartment. This has not posed a noteworthy challenge in the past as analyte-selective sensors have exhibited sufficiently large geometries to ensure that improper sensor insertion can be seen by the naked eye. As sensors have increasingly miniaturized over the years, such as the case with the emergence of microneedle-based analyte-selective sensors, this method has not sufficed. Accordingly, the only viable method capable of addressing this challenge is via electrical interrogation of the said sensor to corroborate successful insertion or to confirm that said sensor remains inserted. Under these embodiments, the user must decide if the reading tendered by the sensor lies within a reasonable or otherwise expected value or range of values. In certain scenarios, a priori knowledge of the reading might be impossible or, at the least, difficult to ascertain.

The technology disclosed herein teaches of a method to circumvent the challenge of assessing if a microneedle-based analyte-selective electrochemical sensor has penetrated/permeated a biological interface and accessed a viable tissue, physiological fluid, or physiological compartment. Specifically, mechanical insertion of at least two spatially distinct electrodes comprising said microneedle-based analyte-selective sensor is attempted in order to penetrate/permeate a biological interface; this is realized by the application of an external physical force by hand or by an external mechanical apparatus.

For the sake of clarity, each microneedle structure features a unique and addressable electrode element and each analyte-selective sensor contained at least two microneedle structures, possessing vertical extent between 20 and 2000 μm, disposed therein. During or after said insertion process, an excitation circuit, embedded in the analyte-selective sensor, applies a fixed or time-varying electrical potential or current to the at least two electrodes located on the at least two microneedle structures. Said fixed or time-varying electrical potential or current interacts at the electrode interface with a tissue, physiological fluid, physiological compartment, or lack thereof, and is thereby modulated in amplitude, frequency, and/or phase, reflecting a change in at least one of a resistance, conductance, capacitance, inductance, or impedance. A measurement circuit subsequently transduces said modulated electrical potential or current generated between the at least two electrodes to the digital domain by means of analog-to-digital conversion.

The quantized, digital value of said modulated electrical potential or current is compared with a pre-programmed threshold or range and a decision circuit determines if the quantized, digital value lies above or below said pre-programmed threshold or within or beyond said pre-programmed range. A YES/NO assessment of sensor insertion into a viable tissue, physiological fluid, or physiological compartment is thus made based on this comparison. Optionally, the analyte-selective sensor system can inform the user via audible, visual, or haptic feedback that said analyte-selective sensor has been satisfactorily inserted and, if not, the system can alternatively direct the user to apply additional external physical force by hand or by an external mechanical apparatus to achieve satisfactory insertion based on the above assessment process. Otherwise, the user can be instructed to remove and re-apply said sensor. In this fashion, a system-directed indication is tendered to the user that provides definitive confirmation of penetration/permeation of a biological interface or membrane and access to a tissue, physiological fluid, or physiological compartment without requiring visual observation of insertion or a user-based assessment of the validity of said analyte-selective sensor readings.

Alternatively, during said insertion process, an excitation circuit, embedded in the analyte-selective sensor, applies an electrical potential, which can also comprise a ground or ‘0’ potential, to the at least two electrodes located on the at least two microneedle structures. When said insertion process is not successful and the electrode interface fails to access said tissue, physiological fluid, or physiological compartment, the excitation circuit is not completed and no (or a negligible amount of) current can flow between said at least two electrodes. Under this scenario, the device cannot activate or power to an ‘ON’ state.

Alternatively, upon suitable insertion of said at least two electrodes located on the at least two microneedle structures into tissue, physiological fluid, or physiological compartment, owing to the high conductivity of said tissue, physiological fluid, or physiological compartment, the said excitation circuit is completed and a current may flow, thereby causing the device to active or power to an ‘ON’ state.

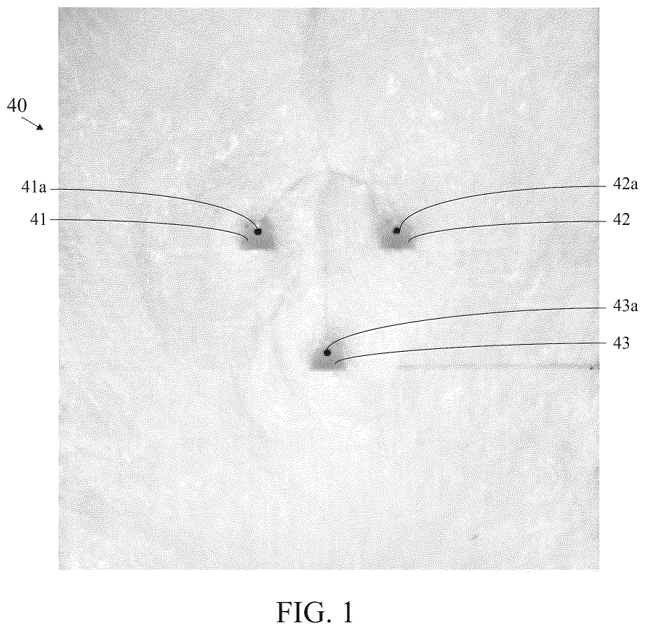

FIG. 1 illustrates an analyte-selective sensor 40 featuring three microneedle structures 41, 42 and 43, each measuring 1200 μm in vertical extent and designed to permeate the stratum corneum to access the interstitial fluid occupying the viable dermis. Each microneedle structure 41, 42, and 43 contains an individually-addressable metal electrode disposed in the circular aperture 41a, 42a and 43a located therein.

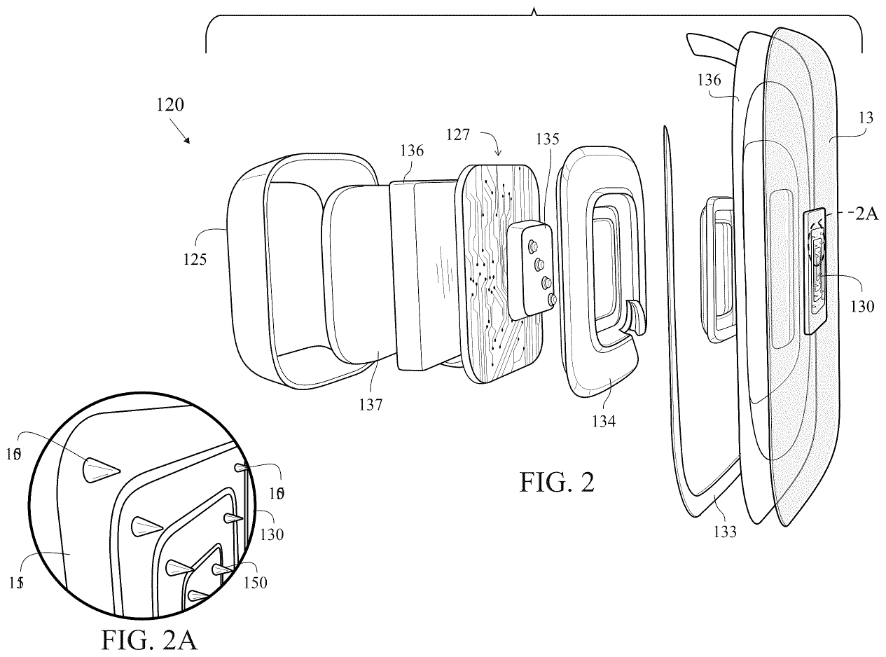

FIG. 2 is an exploded view of a microneedle-based analyte-selective sensor system with the microneedle array 120 and excitation and measurement circuit shown. An expanded view of the microneedle constituents is provided in FIG. 2A. The microneedle biosensing sensor 120 preferably comprises a housing member 125, a back plate 137, an internal pad 136, a circuit board cover 1314, an external pad 133, and adhesive pad, a front panel 131 a microneedle biosensor 130 with microneedles 150, and a printed circuit board 127 containing the electronic circuitry required to transduce biochemical signals to digital data that are wirelessly transmitted to an external device via the embedded wireless transceiver. An electrochemical analog front end preferably includes: a Texas Instruments LMP91000 Sensor AFE System, configurable AFE potentiostat for low-power chemical sensing applications; a Texas Instruments LMP91200 configurable AFE for low-power chemical sensing applications; or an Analog Devices ADuCM350 16-Bit Precision, low power meter on a chip with Cortex-M3 and connectivity. The wireless transceiver is preferably is a BLUEGIGA BLE-113A BLUETOOTH Smart Module, or a Texas Instruments CC2540 SimpleLink BLUETOOTH Smart Wireless MCU with USB.

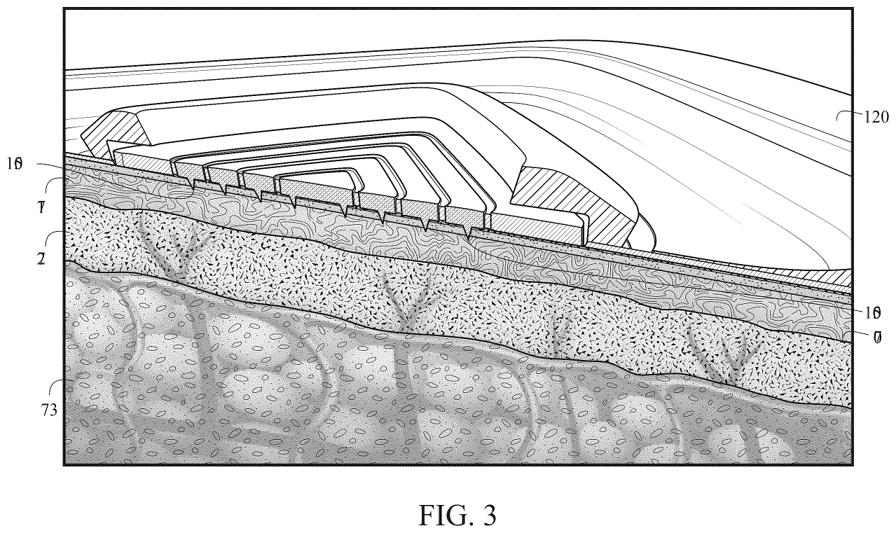

FIG. 3 is a cutaway view of a microneedle-based analyte-selective sensor system 120 with micro-needles 150 successfully penetrating the stratum corneum 70 to access the viable dermis 71.

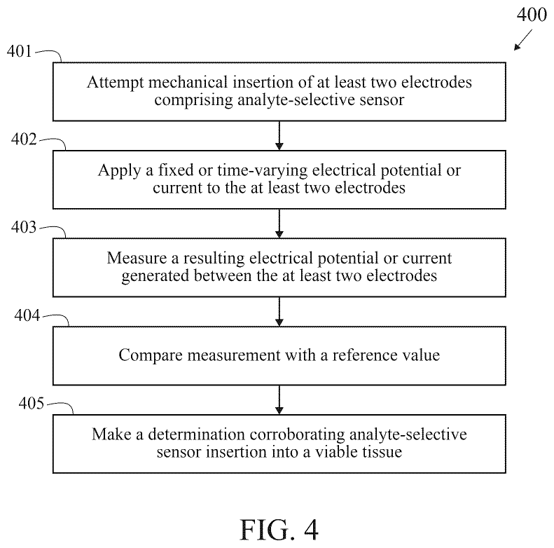

FIG. 4 is a block/process flow diagram illustrating a method 400 to confirm the insertion of a microneedle-based analyte-selective sensor into viable tissue. At block 401, at least two spatially-distinct microneedle structures of an array are inserted into cutaneous tissue of a user. The tissue includes an organ, a membrane, a physiological compartment, and a physiological fluid. The insertion is achieved by means of application of pressure, force velocity or energy to achieve puncture or permeation of the stratum corneum. Each spatially-distinct microneedle structure preferably comprises a single electrode and extends between 200 and 2000 μm from a base of the array. The electrodes comprise metal, semiconductor, or organic surfaces of defined geometry. At block 402, a fixed or time-varying electrical potential or current is applied to the at least two electrodes following the insertion into the cutaneous tissue. The fixed or time-varying electrical potential or current includes a constant voltage, voltage waveform, direct current, alternating current, sinusoidal signal, frequency-dependent signal, impulse, a constant phase signal, and a varying-phase signal. The electrical potential is preferably between zero and one Volt. The electrical current is preferably between one femto-ampere and ten milli-amperes. At block 403, a resultant electrical potential, current value generated between the at least two electrodes is measured. At block 404, the current value is compared to a known reference value to corroborate successful insertion and in situ access to the cutaneous tissue of the user. The reference value includes a voltage, a current, a resistance, a conductance, a capacitance, an inductance, and an impedance. The reference value is set between the preferred electrical potential (one to one Volt) or the preferred electrical current (one femto-ampere and ten milli-amperes). At block 405, a determination is made that the microneedle-based analyte-selective sensor has been successfully inserted into cutaneous tissue.

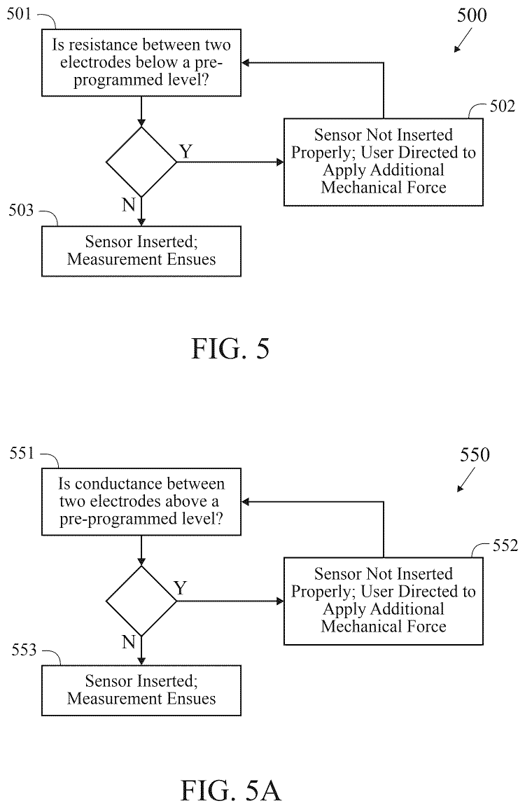

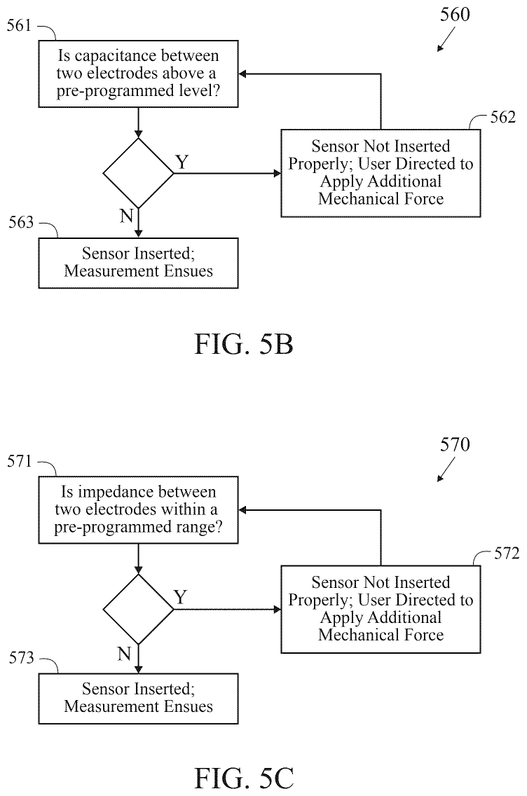

FIGS. 5, 5A, 5B and 5C are a diagrammatic representation of a decision tree delineating the assessment of tissue penetration and hence sensor insertion. The decision tree operates in an embedded system paired to a tissue-penetrating analyte-selective sensor. FIGS. 5, 5A, 5B and 5C illustrate several different electrical methods used to interrogate the electrodes to determine successful insertion into tissue.

As shown in FIG. 5, decision tree 500 begins at block 501 inquiring if a resistance between two electrodes is below a pre-programmed level. If yes, at block 502 the sensor is not inserted properly and the user is directed to apply additional mechanical force. If no, at block 503 the sensor is inserted properly and the measurement is conducted.

As shown in FIG. 5A, decision tree 550 begins at block 551 inquiring if a conductance between two electrodes is above a pre-programmed level. If yes, at block 552 the sensor is not inserted properly and the user is directed to apply additional mechanical force. If no, at block 553 the sensor is inserted properly and the measurement is conducted.

As shown in FIG. 5B, decision tree 560 begins at block 561 inquiring if a capacitance between two electrodes is above a pre-programmed level. If yes, at block 562 the sensor is not inserted properly and the user is directed to apply additional mechanical force. If no, at block 563 the sensor is inserted properly and the measurement is conducted.

As shown in FIG. 5C, decision tree 570 begins at block 551 inquiring if an impedance between two electrodes is within a pre-programmed range. If yes, at block 572 the sensor is not inserted properly and the user is directed to apply additional mechanical force. If no, at block 573 the sensor is inserted properly and the measurement is conducted.

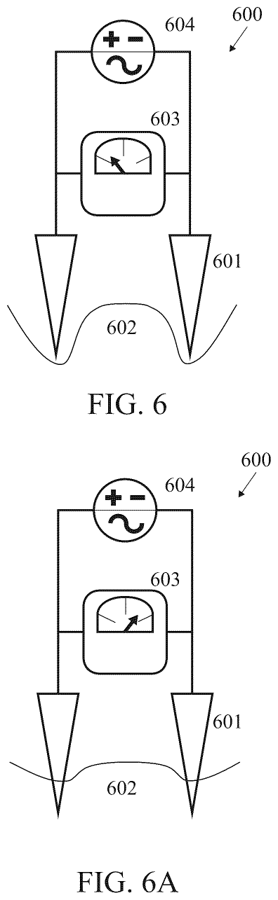

FIG. 6 is a diagrammatic representation of analyte-selective sensor 600 featuring electrodes 601 that have not successfully penetrated/permeated a biological interface/tissue 602, as determined by a measurement circuit 603. The excitation circuit 604 provides the probe signal required to interrogate the electrodes. FIG. 6A is a diagrammatic representation of analyte-selective sensor 600 featuring electrodes 601 that have successfully penetrated/permeated a biological interface/tissue 602, as determined by a measurement circuit 603. The excitation circuit 604 provides the probe signal required to interrogate the electrodes. For the sake of clarity, elements 601, 602 and 603 comprise key constituents of the analyte-selective sensor.

A microneedle-based analyte-selective sensor is a measurement system, containing the below elements, that enables the quantification or assessment of absolute or relative levels of a biological analyte located within a tissue, physiological fluid, or physiological compartment.

Two (or more) electrodes (contained within analyte-selective sensor and designed to reside in the sensing medium) which serving as an electrical-to-biological transducer, provides a means to interface an electronic circuitry with a biological tissue. At least two electrodes are required to form a complete electrical circuit.

An excitation circuit (contained within analyte-selective sensor) provides an electrical excitation signal, stimulus, or probe to interrogate said electrodes.

A measurement circuit (contained within analyte-selective sensor) measures an electrical response generated at the electrode surface in response to said excitation signal, stimulus, or probe.

An embedded decision system makes a determination of sensor penetration/insertion into a viable tissue based on a pre-programmed value, level, or range. Assessment is based on a YES/NO criterion.
McCanna et al., U.S. Pat. No. 9,933,387, for a Miniaturized Sub-Nanoampere Sensitivity Low-Noise Potentiostat System is hereby incorporated by reference in its entirety.
Windmiller et al., U.S. patent application Ser. No. 14/955,850, filed on Dec. 1, 2015, for a Method And Apparatus For Determining Body Fluid Loss is hereby incorporated by reference in its entirety.
Windmiller, U.S. patent application Ser. No. 15/177,289, filed on Jun. 8, 2016, for a Methods And Apparatus For Interfacing A Microneedle-Based Electrochemical Biosensor With An External Wireless Readout Device is hereby incorporated by reference in its entirety.
Wang et al., U.S. Patent Publication Number 20140336487 for a Microneedle Arrays For Biosensing And Drug Delivery is hereby incorporated by reference in its entirety.
Windmiller, U.S. patent application Ser. No. 15/590,105 for a Tissue-Penetrating Electrochemical Sensor Featuring A Co Electrodeposited Thin Film Comprised Of A Polymer And Bio-Recognition Element is hereby incorporated by reference in its entirety.
PCT Publication Wo2018/071265 for an Electro-deposited Conducting Polymers For The Realization Of Solid-State Reference Electrodes For Use In Intracutaneous And Subcutaneous Analyte-selective Sensors is hereby incorporated by reference in its entirety.
Windmiller, U.S. patent application Ser. No. 15/913,709, filed on Mar. 6, 2018 for Methods For Achieving An Isolated Electrical Interface Between An Anterior Surface Of A Microneedle Structure And A Posterior Surface Of A Support Structure is hereby incorporated by reference in its entirety.
Windmiller, U.S. patent application Ser. No. 15/961,793, filed on Apr. 24, 2018 for Heterogeneous Integration of Silicon-fabricated Solid Microneedle Sensors and CMOS Circuitry is hereby incorporated by reference in its entirety.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

1-18. (canceled)

19. An analyte-selective sensor, comprising:
a first microneedle with a first electrode;
a second microneedle with a second electrode; and
electronic circuitry configured to:
apply a first electrical potential to the first electrode and the second electrode;
measure a first electrical value generated between the first electrode and the second electrode;
compare the measured first electrical value to a range of values indicative of successful insertion of the first microneedle and the second microneedle into tissue of a wearer;
apply a second, greater electrical potential to the first electrode and the second electrode when the comparison indicates successful insertion of the first microneedle and the second microneedle into the tissue; and
measure an analyte concentration within physiological fluid based on the second, greater electrical value generated between the first electrode and the second electrode.

20. The analyte-selective sensor of claim 19, wherein the first electrode and the second electrode each comprises an electrochemical sensor.

21. The analyte-selective sensor of claim 19, wherein the first electrode and the second electrode each comprises one or more of a metal, a semiconductor, or an organic surface of a defined geometry.

22. The analyte-selective sensor of claim 19, wherein the first electrical potential is a fixed or time-varying electrical potential comprising a constant voltage, a voltage waveform, a sinusoidal signal, a frequency-dependent signal, an impulse, a constant phase signal, or a varying-phase signal.

23. The analyte-selective sensor of claim 19, wherein the range of values comprises a range of voltage values, current values, resistance values, conductance values, capacitance values, inductance values, or impedance values.

24. The analyte-selective sensor of claim 19, wherein the tissue comprises cutaneous tissue.

25. The analyte-selective sensor of claim 24, wherein the cutaneous tissue comprises the epidermis, dermis, or hypodermis.

26. An analyte-selective sensor, comprising:
a first microneedle with a first electrode;
a second microneedle with a second electrode; and
electronic circuity configured to:
apply, after an attempted insertion of the first microneedle and the second microneedle into tissue of a wearer, a first electrical potential to the first electrode and the second electrode;
measure a first electrical value generated between the first electrode and the second electrode;
compare the measured first electrical value to a range of values indicative of successful insertion of the first microneedle and the second microneedle into the tissue;
instruct the wearer to apply a force to the analyte-selective sensor to attempt re-insertion of the analyte-selective sensor when the comparison indicates successful insertion was not achieved;
apply, after the attempted re-insertion of the analyte-selective sensor, a second electrical potential to the first electrode and the second electrode;
measure a second electrical value generated between the first electrode and the second electrode;
compare the measured second electrical value to the range of values indicative of successful insertion;
apply a third electrical potential to the first electrode and the second electrode when the comparison indicates successful insertion, and
wherein the third electrical potential is greater than the first electrical potential and the second electrical potential.

27. The analyte-selective sensor of claim 26, wherein the first electrode and the second electrode each comprises an electrochemical sensor.

28. The analyte-selective sensor of claim 26, wherein the first electrode and the second electrode each comprises one or more of a metal, a semiconductor, or an organic surface of a defined geometry.

29. The analyte-selective sensor of claim 26, wherein the first electrical potential is a fixed or time-varying electrical potential comprising a constant voltage, a voltage waveform, a sinusoidal signal, a frequency-dependent signal, an impulse, a constant phase signal, or a varying-phase signal.

30. The analyte-selective sensor of claim 26, wherein the range of values comprises a range of voltage values, current values, resistance values, conductance values, capacitance values, inductance values, or impedance values.

31. The analyte-selective sensor of claim 26, wherein the tissue comprises cutaneous tissue.

32. The analyte-selective sensor of claim 31, wherein the cutaneous tissue comprises the epidermis, dermis, or hypodermis.

33. An analyte-selective sensor, comprising:
a first microneedle with a first electrode;
a second microneedle with a second electrode; and
electronic circuitry configured to:
apply a first electrical potential to the first electrode and the second electrode;
measure a first electrical value generated between the first electrode and the second electrode;
compare the measured first electrical value to a range of values indicative of successful insertion of the first microneedle and the second microneedle into the tissue; and
apply a second, greater electrical potential to the first electrode and the second electrode when the comparison indicates successful insertion of the first microneedle and the second microneedle.

34. The analyte-selective sensor of claim 33, wherein the first electrode and the second electrode each comprises an electrochemical sensor.

35. The analyte-selective sensor of claim 33, wherein the first electrode and the second electrode each comprises one or more of a metal, a semiconductor, or an organic surface of a defined geometry.

36. The analyte-selective sensor of claim 33, wherein the first electrical potential is a fixed or time-varying electrical potential comprising a constant voltage, a voltage waveform, a sinusoidal signal, a frequency-dependent signal, an impulse, a constant phase signal, or a varying-phase signal.

37. The analyte-selective sensor of claim 33, wherein the range of values comprises a range of voltage values, current values, resistance values, conductance values, capacitance values, inductance values, or impedance values.

38. The analyte-selective sensor of claim 33, wherein the tissue comprises cutaneous tissue.
